(19) United States
(12) Patent Application Publication
(10) Pub. No.: US 2025/0205297 A1
(43) Pub. Date: Jun. 26, 2025

(54) METHODS AND APPARATUSES FOR THE MANUFACTURE OF CANNABIS-DERIVED PRODUCTS

(71) Applicant: Premium Extracts, Inc., Woodlake, CA (US)

(72) Inventor: Jose L. Rivas, Visalia, CA (US)

(21) Appl. No.: 18/989,355

(22) Filed: Dec. 20, 2024

Related U.S. Application Data
(60) Provisional application No. 63/613,714, filed on Dec. 21, 2023.

(30) Foreign Application Priority Data
May 28, 2024 (GB) 2407539.2

(51) Int. Cl. A61K 36/185; A61K 31/00; A61K 31/01; B01D 3/10; B01D 8/00; B01D 9/00; B01D 11/02

(52) U.S. Cl. CPC A61K 36/3482; A61K 31/01; A61K 31/658; B01D 3/10; B01D 8/00; B01D 9/00; B01D 11/0288; B01D 11/0292; A61K 2236/15; A61K 2236/35; A61K 2236/53; A61K 2236/55; B01D 2009/0086

(57) ABSTRACT

Methods of manufacturing a cannabinoid product can involve extracting from raw cannabis plant material an extract including cannabinoids and terpenes; separating the extract into a cannabinoid rich fraction and a terpene rich fraction; subjecting the terpene rich fraction to heating in a microwave oven thereby to distil terpene components from the terpene rich fraction; collecting the terpene components. In some embodiments, at least one of the terpene components can be recombined with at least one cannabinoid from the cannabinoid rich fraction. Preferably, the heating step is carried out while subjecting the terpene rich fraction to a vacuum. The cannabinoid rich fraction is processed by crystallisation and recrystallisation to produce a purified acidic cannabinoid component, wherein the purified acidic cannabinoid component can remain carboxylated or be decarboxylated is combined with said at least one terpene component. The recombined cannabinoid product can exhibit characteristics closer to those of the original plant.

### CROSS-REFERENCE TO RELATED APPLICATIONS

This application is related to and claims priority benefits from U.S. provisional patent application Ser. No. 63/613,714 filed on Dec. 21, 2023, entitled “Method and Apparatus for the Manufacture of Cannabis-Derived Products”. This application is also related to and claims priority benefits from GB application No. 2407539.2 filed on May 28, 2024.

The '714 and '539.2 applications are hereby incorporated by reference herein in their entireties.

### FIELD OF THE INVENTION

In at least some embodiments, the present invention relates to apparatuses and methods for preparing botanical substances for distillation and/or extraction. More particularly the present disclosure relates to methods and apparatuses for the manufacture of cannabis-derived products, in particular cannabinoid products, and in the preferred embodiments providing rapid and simultaneous decarboxylation and essential oil capture of botanical extracts under vacuum conditions.

### BACKGROUND OF THE INVENTION

A plant medicine's therapeutic activity is attributed to the active constituents it contains. Although there are examples that show the activity of C. Sativa has been linked to specific chemical species, it is also true that the plant's medicinal effect is due to one or more combinations of constituents acting in concert.

Cannabis contains approximately 500 natural compounds. The two classes of compounds of greatest medicinal value are cannabinoids and terpenes. While cannabis may contain literally hundreds of terpenes, only a few have caught the interest of researchers and practitioners, among them: myrcene, linalool, limonene, humulene, pinene and caryophyllene. Until recently, it was thought that terpenes functioned mostly to give cannabis strains their characteristic flavours and aromas. As a consequence, there was little concern about terpene content in a final product because the terpenes were not considered to be important to the medicinal effect of the product.

Recent research has revealed that terpenes play a much greater role in the effect of a particular cannabis strain than originally thought. For example, it has been found that in many situations the interaction between a terpene molecule and a cannabinoid molecule is determinative of the final effect of the relevant strain, with the terpene in practice regulating the medicinal action of the cannabinoid. There is therefore an appreciation of the contribution of terpenes to the medicinal effect produced by a particular cannabis strain.

Over 100 cannabinoids have been identified in cannabis, some of which are psychoactive. Of the cannabinoids, the molecules most studied may be tetrahydrocannabinol (THC) and cannabidiol (CBD), the two cannabinoids accounting for the largest portion of the plant's extract. While THC may account for more than 20% of extract volume in a high-THC strain, CBD levels of over 4% are considered to be high. Recent research has shown CBD to have analgesic, anti-inflammatory, and antianxiety properties without the psychoactive effects associated with THC.

Current methods of preparing cannabinoid agents fail to appreciate the importance of terpenes to the quality and efficacy of the final product. The cannabinoid yield from conventional methods also tends to be sub-optimal. Additionally, the time required for conventional methods places a limit on the amount of extract that can be produced in an economic manner.

### SUMMARY OF THE INVENTION

In at least some embodiments, the present inventions seek to provide improved methods and apparatuses for the manufacture of cannabis-derived products, to improved cannabis-derived products and cannabis-based products, and in some preferred embodiments to methods and systems configured to decarboxylate acidic cannabinoids from a mixture while simultaneously capturing the essential oils under vacuum conditions. In the context of the present disclosure, a cannabis-derived product is to be understood as a product containing one or more components derived from raw cannabis plant material.

According to at least some embodiments of the present disclosure, there is provided methods of manufacturing a cannabis-derived product, comprising the steps of:
extracting from raw cannabis plant material an extract comprising cannabinoids and terpenes;
separating the extract into a cannabinoid rich fraction and a terpene rich fraction;
subjecting the terpene rich fraction to heating in a microwave oven under vacuum conditions thereby distilling the terpene components from the terpene rich fraction while simultaneously decarboxylating the acidic cannabinoids contained in the terpene rich fraction; and
collecting the terpene components.

Optionally, the method comprises recombining at least one of the terpene components with at least one acidic or decarboxylated cannabinoid from the cannabinoid rich fraction.

It has been found that this process enables much greater extraction and recovery of useful components from the raw plant material while using substantially less power, to yield purer products and the ability to reconstitute in useful form a cannabis product having many of the qualities of the original plant material. The resultant product is a marked improvement over prior art cannabis-derived products.

In some embodiments, the methods can comprise recombining at least one of the collected terpene components with at least one acidic or decarboxylated cannabinoid from the cannabinoid rich fraction to produce a cannabinoid product. Thereby, a cannabinoid product having qualities of the original plant material can be selectively can be obtained with faster processing and greater purity than achievable with prior art processes.

Advantageously, the heating step is carried out while subjecting the terpene rich fraction to a vacuum.

Using a vacuum significantly reduces the heat required to extract the terpenes from the terpene rich fraction and/or the extraction time, reducing or eliminating the risk of degradation of the terpenes: while simultaneously decarboxylating the cannabinoids in the terpene rich fraction under vacuum conditions.

In some preferred embodiments, the method comprises the step of collecting the terpene components through at least one cold trap. It may comprise the step of collecting the terpene components through a plurality of cold traps. The plurality of cold traps can be kept at different temperatures. A plurality of cold traps is able to separate different terpene components, allowing selective recombination with the cannabinoid fraction. For example, a first cold trap may be kept at about 5° C., and a second cold trap may be kept at about −50° C., respectively. Further embodiments may comprise first and second colds traps, each cooled in a cooling bath at a temperature of −78° C., for example using a slurry of dry ice in an organic solvent, such as ethanol or acetone.

In some preferred embodiments, the method comprises the step of purifying the cannabinoid rich fraction to produce a purified cannabinoid component, wherein the purified cannabinoid component is combined with said at least one terpene component.

The step of heating the terpene rich fraction in a microwave degrades pesticides or other impurities in the terpene rich fraction. The step of distilling the terpene rich fraction in a microwave degrades pesticides or other impurities in the terpene rich fraction.

Advantageously, the step of extracting from a raw cannabis plant material an extract comprising cannabinoids and terpenes comprises the step of soaking the raw cannabis in a hydrocarbon, preferably butane.

The step of extracting from a raw cannabis plant material an extract comprising cannabinoids and terpenes may comprise the steps of:
processing the raw cannabis plant by shredding and/or finely chopping;
packing the processed raw cannabis plant into one or more vessels; and
soaking the processed raw cannabis in a hydrocarbon.

In some preferred embodiments, the method includes the step of feeding the hydrocarbon after soaking to a collection chamber, removing the hydrocarbon so as to obtain the extract comprising cannabinoids and terpenes. It may also include the step of recirculating the hydrocarbon for reuse in the method.

Advantageously, the hydrocarbon extraction retains the cannabinoids in a carboxylated form.

The step of separating the extract into a cannabinoid rich fraction and a terpene rich fraction can involve resting the extract for a period of time, such as for around 24 hours.

In some preferred embodiments, the method comprises the step of purifying the cannabinoid rich fraction, advantageously by means of one or more crystallization steps. Advantageously, the step of purifying the cannabinoid rich fraction produces tetrahydrocannabinol.

According to another aspect of the present disclosure, there is provided apparatuses for manufacturing a cannabis-derived product, comprising:
an extractor unit configured to extract from raw cannabis plant an extract comprising cannabinoids and terpenes;
a separator configured to separate the extract into a cannabinoid rich fraction and a terpene rich fraction;
a microwave oven configured to heat the terpene rich fraction by microwave radiation thereby to distil terpene components from the terpene rich fraction; and
a collection vessel configured to collect the terpene components.

In some preferred embodiments, the apparatuses comprise a vacuum generator configured to subject the terpene rich fraction to a vacuum and/or at least one cold trap configured to collect the terpene components. In some preferred embodiments, the apparatuses comprise a plurality of cold traps configured to collect the terpene components, the plurality of cold traps being at different temperatures, such as about 5° C., and about −50° C.

In some preferred embodiments, the apparatuses comprise a cannabinoid decarboxylation and purifying station configured to decarboxylate and purify the cannabinoid from the terpene rich fraction. producing a purified and decarboxylated cannabinoid component which may be further refined.

The microwave oven is preferably configured during heating to degrade pesticides or other impurities in the terpene rich fraction and/or to distil the terpene rich fraction to remove pesticides or other impurities originally in the terpene rich fraction.

Advantageously, the apparatuses can comprise a motor configured to rotate the terpene rich fraction during microwave heating in the cannabinoid decarboxylation and purifying station.

There is preferably provided a source of hydrocarbon configured to extract from raw cannabis plant an extract comprising cannabinoids and terpenes by soaking the raw cannabis in the hydrocarbon, the source of hydrocarbon being preferably connected to a vessel configured to hold the raw cannabis plant material.

In some preferred embodiments, the apparatuses can include a collection chamber configured to receive the hydrocarbon after soaking and an extraction mechanism configured to remove the hydrocarbon so as to obtain the extract comprising cannabinoids and terpenes. The extraction mechanism may be a heat source.

There may be provided a crystallisation station configured to crystallise and recrystallise cannabinoid rich fraction so as to produce a purified cannabinoid product.

According to another aspect of the present invention, there is provided methods of manufacturing a cannabinoid product, comprising the steps of:
extracting from raw cannabis plant an extract comprising cannabinoids and terpenes;
separating the extract into a cannabinoid rich fraction and a terpene rich fraction;
subjecting the terpene rich fraction to heating in a microwave under vacuum conditions oven thereby to distil terpene components from the terpene rich fraction while simultaneously decarboxylating the acidic cannabinoids contained in the terpene rich fraction;
collecting the terpene components; and/or
recombining at least one of the terpene components with at least one cannabinoid from the cannabinoid rich fraction to produce a cannabinoid product having a selected number and/or amount of cannabinoids and/or terpenes from the raw cannabis plant.

The cannabinoid compound preferably comprises substantially only or consists of components from the raw cannabis plant.

In at least some embodiments, another aspect of the present invention provides the use of microwave energy in the degradation of pesticides remaining in a terpene rich fraction obtained from raw cannabis plant material.

Other aspects and advantages of the teachings herein will become apparent to the person skilled in the art from the description that follows.

### BRIEF DESCRIPTION OF THE DRAWINGS

Embodiments of the present invention are described below, by way of example only, with reference to the accompanying drawings, in which:

FIG. 1 is a schematic diagram block diagram of a first part of the manufacturing apparatus;

FIGS. 2A and 2B show the extracted cannabis products from the apparatus of FIG. 1, immediately after collection and after a settling time;

FIG. 3 is a schematic diagram block diagram of a second part of the manufacturing apparatus; and

FIG. 4 is a method flowchart illustrating a method of manufacturing a cannabinoid product.

### DETAILED DESCRIPTION OF ILLUSTRATIVE EMBODIMENT(S)

Described below are preferred embodiments of the apparatuses and methods for the manufacture of cannabinoid compounds. Elements of the apparatuses and methods described in the applicant's earlier patent application published as WO-2018/195562 may be used in the apparatuses disclosed herein, and this will be apparent to the person skilled in the art. The methods and apparatuses therefor disclosed in the present application differ significantly from the applicant's earlier disclosed invention, as the skilled person will appreciate, and has significant advantages, including the production of an even more pure end product, faster processing of product and effective treatment and/or removal of pesticides and other unwanted components from the raw material. The removal of pesticides and other unwanted components (impurities) from the raw material and the avoidance of loss of potentially valuable elements from the raw material is considered to be an important advantage. It avoids the loss of potentially valuable constituents from the raw product, the regeneration of a cannabinoid product with more of the original constituents than possible with prior art methods, and the avoidance of non-natural additives of the type used in some prior art production methods. Furthermore, it can lead to a much better final product, including differentiation between final products based on different strains of raw material. In laboratory tests carried out on samples prior to the terpene distillation, that is high terpene extract samples, and samples post distillation, that is cannabis derived terpenes, it has been established that the methods and apparatuses disclosed herein have successfully remediated multiple pesticides through the extraction process.

In further laboratory tests carried out on a sample of high terpene extract, it has been established that the methods and apparatuses disclosed herein demonstrate faster processing and higher yield of terpenes, compared to conventional distillation methods carried out on the same sample of high terpene extract.

The skilled person will appreciate that the description that follows and the accompanying drawings focus on the main components and elements of the apparatuses and methods and omit inessential detail that is commonplace in the art and that would be immediately apparent to the skilled person. The drawings are therefore largely schematic for this purpose and intended to highlight the principal components of the apparatuses and elements of the methods and processes.

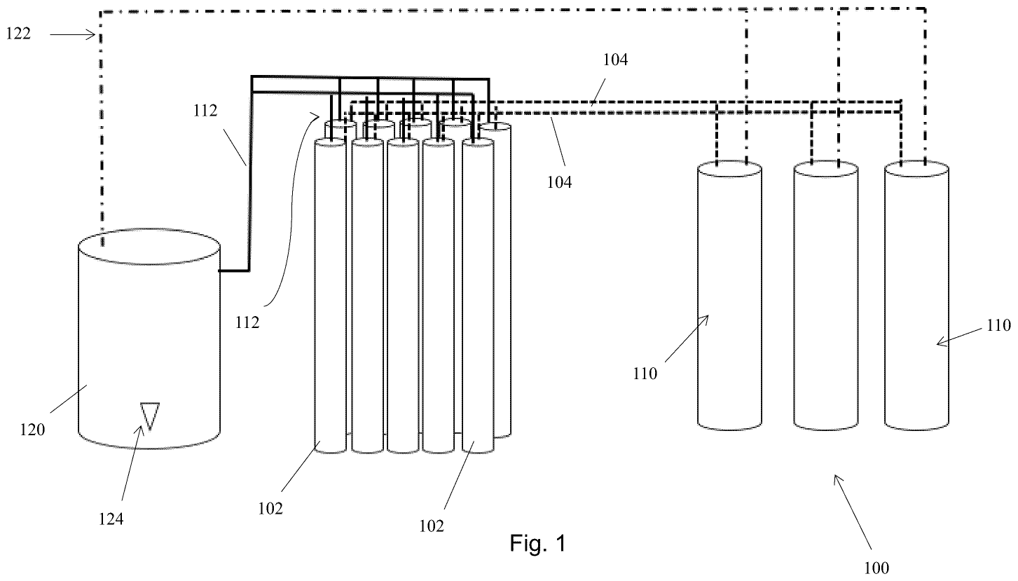

The first stage of the process involves the extraction of cannabinoids and terpenes from the raw material. In some preferred embodiments, this is done by hydrocarbon extraction, preferably using an apparatus as shown in FIG. 1.

The apparatus 100 includes, in at least some embodiments, a plurality of canisters or vessels 102 configured to hold raw material, in this case cannabis plant material. Fresh cannabis plants may be initially processed by being shredded and/or finely chopped, which allows the raw material to be closely packed into the vessel 102 and allows uniform exposure of the fresh plant material to the processing hydrocarbons.

In other embodiments, there may be provided a single vessel 102. However, a plurality of vessels allows the processing of more raw bulk material, in the preferred embodiments more raw cannabis plant material.

The vessels 102 include inlet conduits 104 or tubes coupled to one or more sources 110 of a hydrocarbon fluid. The preferred hydrocarbon is butane, although the skilled person will know that other hydrocarbons could be used, such as: methanol, ethanol, isopropanol, hexane, pentane, butane, propane, naphtha, chloroform, and so on.

In the embodiment shown in FIG. 1, there are provided a plurality of sources or vats 110 of hydrocarbon. A plurality is preferably provided to enable the storage of hydrocarbons at different temperatures and/or different hydrocarbons, for use with different strains or types of raw material and/or to treat the same raw material bulk to hydrocarbons at different temperatures and/or to different hydrocarbons. So doing can ensure optimum extraction of the desired components from the raw material, in the preferred embodiments, extraction of cannabinoids and terpenes from the raw cannabis plant.

The skilled person will appreciate that in other embodiments there may be provided a single source 110 of hydrocarbon.

The hydrocarbon is fed into the vessels 102 through the conduits 104, in practice to soak the raw material (cannabis in this example) with the hydrocarbon. The hydrocarbon acts to dissolve from the raw plant material the desired constituents, specifically cannabinoids and terpenes in this example. The process is in the preferred embodiments run between −40° C. and −75° C., at pressures between 20 PSI and 100 PSI, and exposed for solvent for 2 to 20 minutes.

Each vessel 102 also comprises an outlet conduit 112 which is coupled to a collection chamber 120. Under pressure preferably between 20 PSI and 100 PSI, the hydrocarbon passes from the vessels 102 to the collection chamber 120, taking with it the dissolved cannabinoids and terpenes.

This part of the process takes advantage of hydrocarbon extraction in which the cannabis raw material remains in an acidic or carboxylated form. This differs from prior art methods, for example as described in the applicant's earlier WO-2018/195562. In at least some preferred methods, the raw cannabis remains in its acidic form, that is non-decarboxylated, during cannabinoid extraction. It is desired that the terpenes remain in mix and by these methods the terpenes in the original raw material are captured through the hydrocarbon extraction.

The hydrocarbon, with the entrained cannabinoid and terpene compounds, flows from the vessels 102 into the collection chamber 120.

The collection chamber 120 is a distillation device for the distillation of butane of other hydrocarbon from the mix fed into the chamber 120. The skilled person will appreciate that the butane may be kept under pressure and/or at a cold temperature below its boiling point and then released by evaporation by raising its temperature and/or pressure. It will be appreciated that for this purpose the collection chamber 120 may be coupled to a heating/cooling unit and/or to a pressure control and/or to a solvent pump unit for effecting the distillation.

The collection chamber 120 includes a feedback conduit 122 that feeds butane retrieved from the distillation back to the original source vessel/vessels 110 from which it originate(s), for reuse.

It will be appreciated that the conduits 104, 112 and 122 shown in FIG. 1 may be individual conduits from one vessel to another, or may be in the form of a manifold in which multiple vessels are connected together, in dependence upon the end use of the vessels, that is whether they contain the same materials and are used for increasing production capacity, or different materials or materials in different states, for different production purposes. It will also be appreciated that the conduits 104, 112, 122 will typically also be valved, most advantageously being controlled by an automated control unit or computer.

The collection chamber 120 also includes an outlet 124 for dispensing the extracted components from the raw material, termed herein the hydrocarbon crude. Typically, this will be in the form of a viscous liquid.

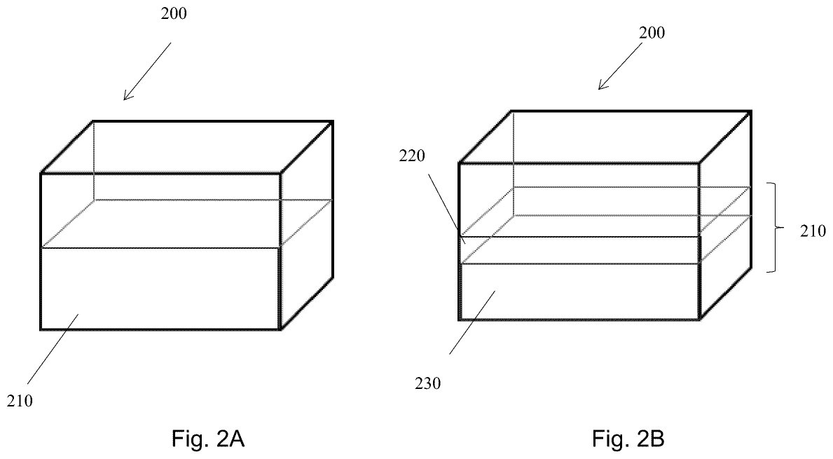

Referring now to FIGS. 2A and 2B, these show in very schematic form a collection vat 200 into which the hydrocarbon crude 210 can be collected. When first collected the crude will be in a supersaturate homogeneous mix, as shown in FIG. 2A. However, over time, typically 1 to 24 hours or so, the crude separates and crystalizes into two layers 220 and 230. The upper, lighter, layer is generally less viscous, and oily, and contains the majority of the terpene compounds from the hydrocarbon crude as well as acidic cannabinoids. It will typically also include impurities such as pesticides and so on.

The bottom layer, which is more viscous, grainy or sandy in consistency, 230 contains the majority of the cannabinoids. This layer is typically substantially solid and is formed of a much purer fraction of cannabinoids. This layer can be purified into a white powder. The purification process, to remove contaminants from the cannabinoid layer, typically raises the purity from around 80-85% tetrahydrocannabinolic acid (THCA) to around 98% tetrahydrocannabinolic acid (THCA). This can them be purified further in another solvent through crystallization and recrystallization components to virtually pure tetrahydrocannabinol, which is typically in the form of a white powder. The crystallisation process of the cannabinoids can get to a very pure form of THC, up to 99.9% purity. The resultant product can then be melted, decarboxylated, under vacuum, to create an oil of extreme purity. The person skilled in the art will be familiar with suitable purification processes.

### Winterization

After the terpene extraction a decarboxylated crude extract having a high concentration of cannabinoids, may also contain residual terpenes that were not extracted during the initial process. The crude extract may also contain a number of undesirable components such as lipids, waxes, pigments, or other such plant material. An additional refinement process serves to greatly reduce the level of these undesirable components and prepare the crude extract for downstream processing.

Many of the impurities remaining in the extract may be longer-chain hydrocarbons having a relatively high melting point. Thus, a refining procedure to remove the long-chain impurities involves dissolving the crude extract in a pure alcohol preferably MeOH, chilling the alcohol solution and filtering it to remove the precipitated long-chain impurities. Additionally, treating the crude extract with alcohol, freezing and filtering have the added benefit of removing more of the residual terpenes and further concentrating the cannabinoid fraction.

The initial step in the winterization process involves re-dissolving the crude extract in alcohol. In embodiments, the alcohol may be at room temperature. In embodiments, to facilitate dissolving the crude oil in the alcohol, the alcohol may be heated to a temperature slightly above room temperature (RT), for example 30° C. While a lower temperature helps to ensure a higher cannabinoid concentration in the final product, in certain applications, it may be desirable to heat the alcohol considerably above RT, for example, as high as 45° C.

In embodiments, the crude oil and the alcohol may be combined in a predetermined ratio, for example 10 ml of alcohol for each gram of extract. Choice of the ratio of oil to alcohol is driven by a number of factors, such as lipid and cannabinoid concentration of the starting material.

After the alcohol and the crude oil are combined, the mixture may be stirred, either manually, using a laboratory implement such as a spatula, or by using an automated stirring device, such as a magnetic stirrer. After the crude oil is fully dissolved in the alcohol, the solution is held at an approximate temperature of −20° C. for a period of approximately 24 hours. The solution is then passed through a filter having a 25-micrometre pore size to remove precipitated solids.

The solution may again be refrozen to −20° C. and held for 24 hours, after which it may be passed through a 2.5 micrometre filter.

The solution may again be refrozen to −20° C. and held for 24 hours, after which it may be passed through a 0.2 micrometre membrane filter.

It will be appreciated that the duration of the freezing periods may be reduced by subjecting the solution to lower temperatures. For example, if the solution is frozen using, for example, dry ice, the freezing time may be reduced. The freezing point of the solvent and/or the solute, would, of course, impose a lower limit on the freezing temperature.

The waxes removed from the oil as a result of winterization may be discarded, however they may also be recovered and diverted into other processes/products, skin care additives or personal lubricants, for example.

After the waxes, pigments and plant material are removed during the winterization process, the alcohol may be purged from the solution, leaving a winterized extract. In embodiments, using a vacuum and a vacuum oven, the boiling point of the alcohol can be reduced to well below room temperature. In embodiments, the alcohol may be purged using a rotary evaporator.

### Wiped-Film Distillation

The final stage of at least some preferred methods utilizes a wiped-film distillation technique to purify the winterized material into a distillate containing highly concentrated cannabinoids in ratios similar to those found in the original plant material. Purity is typically greater than 98% total cannabinoids and may be as high as 99.9% pure cannabinoids.

The winterized extract typically has a thick, syrupy consistency. Additionally, because of the high cannabinoid content, the extract is very heat-sensitive. Thus, it is desirable, at all stages of processing to minimize, or at least reduce, exposure of the extract to higher temperature. A conventional still-pot distillation provides a number of disadvantages for a heat-sensitive product such as the presently described extract. Chief among these is the long residence time necessitated by the conventional pot distillation. As indicated, the extended thermal exposure in a batch process system degrades the quality of the extract. In particular, exposure of the cannabinoids in the extract to the extended heat in a conventional pot distillation system causes a significant drop in the cannabinoid concentration of the final product.

Another disadvantage of a conventional pot distillation system is that a large volume of product is lost due to the extract fouling the equipment as a result of its sticky, viscous consistency. As a matter of fact, the cannabis extract can foul the equipment so badly that it has to be discarded frequently.

Another disadvantage of a conventional pot distillation approach is the very slow rate of processing. The speed with which a quantity of extract can be distilled using a wiped-film approach produces a distinct business advantage for commercial producers.

Finally, as indicated above, the wiped-film approach allows the production of an extract of exceptional purity and potency.

In embodiments, this final distillation may utilize a wiped-film evaporator and vacuum distillation. In embodiments, this final distillation may utilize a short-path wiped film evaporator having a condenser that is located inside the evaporator body.

A wiped film evaporator can provide the short residence time needed and an open, low pressure drop configuration, allowing continuous, reliable processing of heat sensitive, viscous, or fouling materials such as cannabis extract, without product degradation.

In a wiped-film evaporator (WFE), the product is fed into the top of a cylinder and evenly dispersed by a distributor. Heat is applied to the exterior surface of the cylinder, for example by a heated jacket. As the liquid film runs down the inside surface of the cylinder, a rotating wiper system spreads, agitates and moves the product down and off of the heated cylinder wall in a matter of seconds. Heat transfer under vacuum conditions causes the product to evaporate at a greatly reduced temperature. Evaporated product is allowed to pass through a liquid-vapor separator, while droplets of unevaporated product are thrown back to the heated surface. The vapor condenses on a condenser enclosed within the cylinder and exits the WFE through a distillate outlet.

In embodiments, condensation may take place in a condenser located outside of the evaporator.

The wiped-film distillation may be carried out using any of a number of commercially available wiped-film, short-path stills. For example, in some embodiments, the wiped-film distillation may be carried out using a still such as the Pope Wiped-Film Molecular Still, manufactured by POPE SCIENTFIC, Inc., Saukville, WI.

In embodiments, the wiped-film distillation may be carried out using a still such as a Thin-Film, Short-Path Evaporator manufactured by LCI, Inc. Charlotte, NC.

In embodiments, the wiped-film distillation may be carried using a still such as a Short-Path Distillation Plant manufactured by ROOT SCIENCES, Inc., Seattle, WA.

Any of the foregoing systems are vacuum distillation systems that embody the use of a wiped-film (aka “thin-film”) evaporator having an incorporated condenser, which greatly reduces the amount of time the product is exposed to heat to no more than a few seconds. The provision of a short path between the evaporator and the condenser allows the pressure within the system to be kept at a level that approximates the vapor pressure of the cannabinoid fraction, allowing the cannabinoid fraction to evaporate rapidly with minimal application of heat, thereby preserving the cannabinoids.

While several commercially available systems have been described herein above, one of ordinary skill will readily realize that a system for wiped-film distillation system embodying the same operative principles as the named systems may be obtained from other sources or it may be constructed from readily available components, either of which embodiments are entirely consistent with the scope of the present disclosure and the attached claim.

As described above, the sub-process may include at least the steps of:
introducing liquid extract into a wiped-film evaporator (WFE) under vacuum conditions;
dispersing the liquid extract on the heated interior surface of the WFE by means of a wiper system;
separating extract evaporated by a combination of vacuum conditions and heat transfer from liquid extract;
condensing evaporated extract; and
collecting distillate.

The methods for preparing concentrated extracts of cannabis may be deployed at any scale.

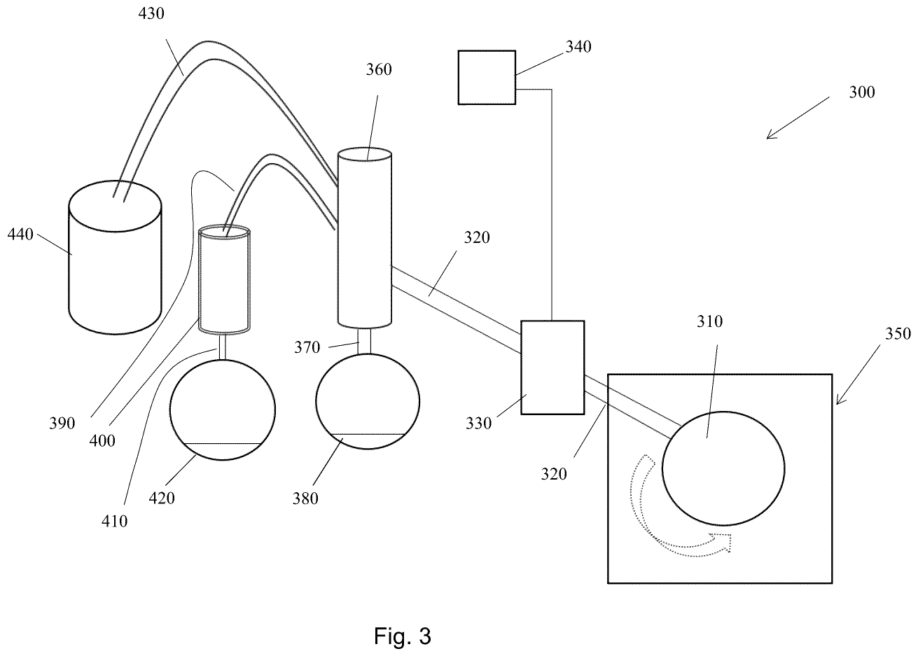

Referring now to FIG. 3, this shows in schematic form a preferred embodiment of an apparatus for extracting the terpenes from the lighter fraction/layer 220 of the crude 210.

The terpene extractor apparatus 300 comprises a vessel 310 preferably of spherical form with an outlet conduit 320. The conduit 320 is connected to a motor unit 330 electrically connected to a speed control unit 340. The vessel 310 is disposed in a microwave over 350, which may be a 1,000 or 1,100 Watt oven, although its power will be dependent on the size of the vessel 310 and desired heating parameters. The conduit 320 is rotatably located in a bushing or similar component in a wall of the oven 350.

The conduit 320 connects to a first cold trap 360, of known form. The cold trap 360 is coupled by an outlet conduit 370 to a first terpene collection vessel 380, described in further detail below. A second cold trap 400 is coupled to an upper portion of the first cold trap 360 by a conduit 390 and has an outlet conduit 410 that couples to a second terpene collection vessel 420.

A vacuum pump 440, coupled by a conduit 430, is connected to the cold trap 360 and therefrom to the entire interior volume of the apparatus, and especially to the vessel 310. The vacuum pump 440 maintains the interior of the apparatus, and hence the materials therewithin, under vacuum. This significantly reduces the required operating temperature of the apparatus and as a consequence significantly reduces the risk of degradation of the material, particularly the terpenes, leading to significantly greater yield of usable terpenes.

The vacuum may be in a range of less than 0.1 Pa to as much as 266 Pa.

The coupling of the second cold trap 400 to the first cold trap 360 creates a closed loop system, useful in maintaining the entire apparatus under vacuum.

While the embodiment shown in FIG. 3 comprises two cold traps 360, 400 with their respective terpene collection vessels 380, 420, in some embodiments there may be only one cold trap and terpene collection vessel, while in other embodiments there may be more than two. The cold traps 360 and 400 are preferably kept at different temperatures, designed to collect different terpenes. For example, the cold trap 360 may be kept at room temperature, while the cold trap 400 may be kept at a much colder temperature, for example by being immersed in vessel of dry ice.

The different cold traps could be kept a variety of temperatures in dependence on the nature of the crude oil and the terpenes it contains. In some examples, on cold trap could be set at 100° C., another at 75° C., and even lower temperatures, such as 5° C., down to −50° C.

In the example of FIG. 3, the cold traps 360 and 400 are preferably both cooled to be at 5° C., and −50° C. respectively.

Cooling of the cold traps 360, 400 can be by immersing these into a container of cooling medium, or by having jacketed vessels, in which an outer jacket can be filled with cooling fluid, or a mixture of the two.

The different cold traps enable the apparatus to distil out or fractionate out multiple components based on evaporation point of the terpenes in the oil.

In use, oil from the layer 220 obtained from the process of FIG. 1 is poured into the heating flask/vessel 310 (which for the sake of convenience can be decoupled from the conduit 320 and removed from the oven 350). The oven is then operated in order to heat the oil, while rotating the vessel 310 by means of the motor 330. This rotation ensures even heating throughout the volume of oil in the vessel 310.

In practice, from 1 kg to 1.5 kg of oil would be loaded into the vessel per cycle.

As the oil is heated, the terpenes will evaporate, passing through conduit 320 into the first cold trap 360 and through the conduit 390 into the second cold trap 400.

It has been found that these heating apparatuses and methods, especially when carried out under vacuum, provides much more efficient distillation of the terpene fractions in the oil and can also be operated at lower temperatures, which ensures that the terpenes are not degraded by the distillation process.

The process disclosed herein can be carried out in significantly less time than prior art processes, especially those that require the target compound to be heated for a much longer period, which can thermally degrade the cannabinoids. The process disclosed herein, it has been found, can take 20 minutes or so, compared to several hours with conventional methods.

Distillation occurs because the terpenes have a lower boiling point than the other components in the oil, including remaining cannabinoids, pesticides and so on. Thus, when heat energy and vacuum energy by a reduction vacuum are applied to this system, as heat increases various chemicals in the oil volatilise, terpenes being the ones of interest.

The heating of the terpene rich fraction also provides for simultaneous decarboxylation of the acidic cannabinoids contained in the terpene rich fraction, which are collected in the cold traps.

In general terms, it has been found that this heating system can recover anywhere from 30-99% of the total available terpenes in the oil, with yields of 20-50% being very achievable depending on the quality of the starting material.

The volatilised terpenes are then condensed in each cold trap and drip into the respective receiving vessel 380, 420. As the cold traps 360, 400 can be of known design and form, they are not described in detail herein.

Lighter boiling point terpenes will get trapped by the second cold trap 400. The different temperature cold traps provide the benefit of capturing at least two slightly different fractions of terpenes of interest.

As explained above, the recovered terpenes are then used, in a subsequent step of the process, to recombine them with the extracted cannabinoids. Preferably, the same terpenes, or a selection of these, is recombined with the purified cannabinoid as those from the original raw material. Doing this in practice reconstructs many of the characteristics of the original product, but in a useful and legal form. For example, recovered terpenes can be used to create a cannabis oil which is of unusual potency and flavour, as a recreational product, and/or as a pharmaceutical product.

In terms of pharmaceutical products, there are various cannabinoids or cannabis variables which have been shown to have medicinal effects. Those medicinal effects have been described to be part of what is colloquially known as the entourage effect. The entourage effect is an effect that is obtained not simply from one purified cannabinoid but, it has been found from a subset of cannabinoids and terpenes that are found within the context of the plant. Each individual plant or strain is unique in its own right. In at least some embodiments, disclosed methods can purify both classes of the most desirable compounds (cannabinoids and terpenes) to generate the entourage effect and create a very pure substance which then can be administered as an emolument or in any other suitable form.

In terms of recreational use, each cannabis strain has a unique aroma, being something that is valued.

The methods taught herein provide an ability to concentrate various terpene oils and then combine them to create recreational products in which the flavour of the original strain is exhibited in a more purified form, yet as a natural flavour and not artificially synthesised.

This is considered to provide a significant improvement over prior art methods that are unable to recover the original terpenes in a usable form and which therefore must rely on blending the cannabinoids with non-original constituents, such as other terpenes and fillers (glycol, glycerine for example). Doing so loses the qualities of the original material and creates a generic end product with little unique value.

In practice, only a percentage of the original terpenes is recombined into the cannabinoid fraction, be it a percentage of the number of terpenes originally present and/or a percentage in terms of amount or concentration. This can be chosen during the manufacture process, in dependence upon the desired end characteristics of the product, such as potency, flavour, and so on.

In at least some embodiments, the method therefore enables the use of the same ingredients of the original plant, but with the ability, having extracted the original terpenes, to accurately dose their recombination.

In summary, the important components from the original raw plant material can be extracted, purified and then recombined into formulations of cannabinoid products of unusual purities and potencies and also unique variations of those that encompass the full flavour aspect and aroma of each cannabis strain extracted. Trapping the unique qualities of each could be seen as similar to the wine, scotch and other markets in terms of how different products are distinguished and the value of the end product.

For use as a pharmaceutical product, the disclosed manufacturing systems and methods are able to isolate the cannabinoid components and also the terpenophenolic compounds and then recreate a purified substance that could then be used as a pure pharmaceutical, as an example.

As discussed, the apparatuses and methods disclosed in the present application, especially when carried out under vacuum, provide much more efficient distillation of the terpene fractions in the oil.

In a specific example, the efficiency of the methods and apparatuses disclosed herein were assessed in comparison to distillation by a conventional process. A sample of high terpene extract was prepared in accordance with the method described above with reference to FIGS. 2A and 2B. Namely, a hydrocarbon crude was prepared by hydrocarbon extraction of a raw cannabis sample and the lighter fraction (HTE) was isolated. A first batch of the HTE sample was treated according to the method described above with reference to FIG. 3. A second batch of the HTE sample was treated using a commercially available terpene distillation kit (10L terpene distillation kit, manufactured by Better Flange Spd LLC, El Cerrito, CA; SKU: 364215376135191). Both batches of HTE was preheated to 60° C. prior to distillation.

BATCH 1: microwave distillation of 1407 grams of HTE was carried out in a microwave oven at 1000 watts under vacuum conditions. The distilled terpene product was collected in a terpene collection vessel cooled to approximately −78° C. by immersing the terpene collection vessel in a cooling bath containing a slurry of dry ice and ethanol. Distillation was completed in 35 minutes. A total yield of 298 grams of terpene essential oils was obtained at a rate of approximately 8.5 grams per minute.

BATCH 2: convention distillation of 1312 grams of HTE was carried out at 160° C. under the same vacuum conditions as Batch 1. A vessel containing the HTE was heated to 160° C. in a heating mantel. The distilled terpene product was collected in a terpene collection vessel cooled to approximately −78° C. by immersing the terpene collection vessel in a cooling bath containing a slurry of dry ice and ethanol. Distillation was stopped after 35 minutes, yielding 24.4 grams of terpene essential oils at a rate of approximately 0.7 grams per minute.

Accordingly, it was found that treatment of the HTE by the methods and apparatuses of the present disclosure resulted in a significant (approximately 12-fold) increase in efficiency compared to known methods.

However, in addition to improved efficiency, there is another significant advantage that has been discovered with the methods and apparatuses taught herein, which is a markedly greater recovery of usable components from the original plant material, particularly usable terpenes.

There is a problem within the cannabis industry, that the cannabinoid rich fraction and the terpene rich fraction from the extraction have a tendency to quantify pesticides, heavy metals, and other unwanted impurities. It has been a problem in the art removing these from the terpenes rich layer and frequently there is a fraction of pure cannabinoids which are compliant within the context of most legal markets, and then a terpene fraction which is unsellable. The methods and apparatuses taught herein, however, are able to isolate out the terpenes in a non-contaminated form and then usable to create a product with more value.

It has been found that the process of heating the terpene rich oil in a microwave oven removes and/or degrades any pesticides that remain inside what would then be decarboxylated terpene. While it is not clear precisely what mechanism is removing or degrading the pesticides and other impurities, the end result is noticeable. It is possible that the systems and apparatuses cause thermal degradation of the pesticides and other impurities. The other it is possible that there is a degree of distillation. One theory is that the microwave energy itself creates a molecular situation that degrades these impurities while not degrading the cannabinoid/terpene constituents.

Prior art systems rely on indirect heating methods, for example a heating mantle or the like. The system taught herein, on the other hand, uses direct energy through the microwaves themselves. Even though the process is carried out under a deep vacuum, and therefore lowering boiling points, microwaves have very interesting properties with how they heat objects, particularly through excitation of molecules of the volume being heated.

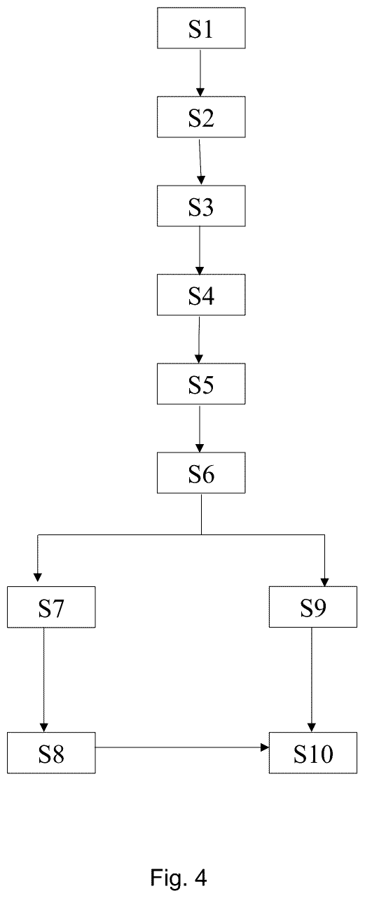

Referring now to FIG. 4, this shows an exemplary method flowchart according to the present disclosure comprising the following steps:
S1: Shredding/chopping raw cannabis plant.
S2: Packing processed raw cannabis plant into one or more vessels.
S3: Soaking processed raw cannabis in hydrocarbon (e.g. butane).
S4: Feeding hydrocarbon to collection chamber and removing hydrocarbon to obtain extract comprising cannabinoids and terpenes.
S5: Resting extract for period of time (e.g. around 24 hours) to separate extract into cannabinoid rich and terpene rich fractions.
S6: Separating cannabinoid rich fraction and terpene rich fraction.
S7: Heating terpene rich fraction in microwave under vacuum conditions.
S8: Collecting terpene components in one or more cold traps.
S9: Purifying cannabinoid rich fraction to produce purified cannabinoid component.
S10: Recombining at least one collected terpene component with at least one cannabinoid component.

It will be appreciated that any of steps S1 to S10 may be carried out or modified as described substantially above in relations to FIGS. 1 to 3.

While the foregoing written description enables one of ordinary skill to use the methods herein described, those of ordinary skill will understand and appreciate the existence of variations, combinations, and equivalents of the specific embodiments, methods, and examples herein. The specification should therefore not be limited by the above-described embodiments, method, and examples, but by all embodiments and methods within the scope of the claims.

The disclosure in the Abstract accompanying this application is incorporated herein by reference.

While particular elements, embodiments and applications of the present invention have been shown and described, it will be understood, that the invention is not limited thereto since modifications can be made without departing from the scope of the present disclosure, particularly in light of the foregoing teachings. For example, the numerous embodiments demonstrate that different combinations of components are possible within the scope of the claimed invention, and these described embodiments are demonstrative and other combinations of the same or similar components can be employed to achieve substantially the same result in substantially the same way. Further, all of the claims are hereby incorporated by reference into the description of the preferred embodiments.

What is claimed is:

1. A method of manufacturing a cannabis-derived product comprising the steps of:
extracting from a raw cannabis plant material an extract comprising cannabinoids and terpenes;
separating the extract into a cannabinoid rich fraction and a terpene rich fraction;
subjecting the terpene rich fraction to heating in a microwave oven under vacuum conditions thereby distilling an at least one terpene component from the terpene rich fraction while simultaneously decarboxylating the acidic cannabinoids contained in the terpene rich fraction; and
collecting said at least one terpene component.

2. The method of claim 1 further comprising recombining said at least one terpene component with at least one acidic or decarboxylated cannabinoid from the cannabinoid rich fraction to produce a cannabinoid product.

3. The method of claim 1 further comprising the step of collecting the terpene components through a plurality of cold traps.

4. The method of claim 1 further comprising the step of purifying the cannabinoid rich fraction to produce a purified cannabinoid component, wherein the purified cannabinoid component is combined with said at least one terpene component.

5. The method of claim 1 further wherein the step of extracting from the raw cannabis plant material the extract comprising cannabinoids and terpenes comprises the step of:
soaking the raw cannabis plant material in a hydrocarbon.

6. The method of claim 1 wherein the step of extracting from the raw cannabis plant material the extract comprising cannabinoids and terpenes comprises the steps of:
processing the raw cannabis plant material by shredding and/or finely chopping;
packing the processed raw cannabis plant into one or more vessels; and
soaking the processed raw cannabis in a hydrocarbon.

7. The method of claim 5 further comprising the step of feeding the hydrocarbon after soaking to a collection chamber; and
removing the hydrocarbon to obtain the extract comprising cannabinoids and terpenes.

8. The method of claim 7 further comprising the step of recirculating the hydrocarbon for reuse in the method.

9. The method of claim 5 further wherein the hydrocarbon extraction retains the cannabinoids in a carboxylated form.

10. The method of claim 1 further comprising the step of decarboxylating and purifying the cannabinoid rich fraction,
wherein the step of purifying the cannabinoid rich fraction comprises one or more crystallization steps.

11. The method of claim 10 wherein the step of purifying the cannabinoid rich fraction produces tetrahydrocannabinol.

12. The method of claim 1 further comprising recombining at least one of the collected terpene components with at least one acidic or decarboxylated cannabinoid from the cannabinoid rich fraction to produce a cannabinoid product having a selected number and/or amount of cannabinoids and/or terpenes from the raw cannabis plant material,
wherein the extract comprising cannabinoids and terpenes is extracted from the raw cannabis plant material.

13. The method of claim 1 wherein the cannabis-derived product is a cannabinoid product comprising substantially only components from the raw cannabis plant material or consists only of components from the raw cannabis plant material.

14. An apparatus for manufacturing a cannabis-derived product, comprising:
an extractor unit configured to extract from a raw cannabis plant material an extract comprising cannabinoids and terpenes;
a separator configured to separate the extract into a cannabinoid rich fraction and a terpene rich fraction;
a microwave oven and vacuum pump configured to heat the terpene rich fraction by microwave radiation under vacuum conditions thereby to distil terpene components from the terpene rich fraction while simultaneously refining and decarboxylating the cannabinoids found in the terpene rich fraction; and
a collection vessel configured to collect the terpene components.

15. The apparatus of claim 14 further comprising a plurality of cold traps configured to collect the terpene components.

16. The apparatus of claim 14 further comprising a cannabinoid decarboxylation and purifying station configured to decarboxylate and purify the cannabinoid rich fraction from the terpene rich fraction to produce a purified and decarboxylated cannabinoid component.

17. The apparatus of claim 14 further comprising a source of hydrocarbon configured to extract from the raw cannabis plant material an extract comprising cannabinoids and terpenes by soaking the raw cannabis plant material in the hydrocarbon.

18. The apparatus of claim 14 wherein a source of hydrocarbon is connected to a vessel configured to hold the raw cannabis plant material.

19. The apparatus of claim 18 further comprising:
a collection chamber configured to receive the hydrocarbon after soaking; and
an extraction mechanism configured to remove the hydrocarbon to obtain the extract comprising cannabinoids and terpenes.

20. The apparatus of claim 14 further comprising a crystallisation station configured to crystallise and recrystallise cannabinoid rich fraction so as to produce a purified cannabinoid product.
